(19) United States
(12) Patent Application Publication
(10) Pub. No.: US 2009/0298152 A1
(43) Pub. Date: Dec. 3, 2009

(54) PROCESS FOR THE SIMULTANEOUS REMEDIATION AND PRODUCTION OF FUEL FROM FRACTIONALIZED WASTE AND VIRGIN MATERIALS THROUGH THE USE OF COMBINATIVE BIOREACTOR AND CATALYTIC METHODOLOGY

(76) Inventor: Dennis M. Danzik, Scottsdale, AZ (US)

Correspondence Address: RATNERPRESTIA, P.O. BOX 980, VALLEY FORGE, PA, 19482, US

(21) Appl. No.: 12/130,046

(22) Filed: May 30, 2008

(51) Int. Cl. C12P 1/00

(52) U.S. Cl. 435/170; 435/171

(57) ABSTRACT

A process for the production of fuel or biodiesel, and use of the process to fuel an on-site engine. The process includes introducing an effluent stream into a first vessel, the effluent stream containing solids and at least one of vegetable oil and petroleum oil. The process includes heating the effluent to lower its viscosity in the first vessel, separating at least some of the solids from the heated effluent to form a reactant stream, introducing the reactant stream to a bioreactor, introducing at least one remediation agent into the bioreactor, heating the reactor contents to a reaction temperature, and allowing sufficient time to pass such that the reactant stream yields a fuel product.

### FIELD OF THE INVENTION

The present invention relates fuel production, and more specifically to biological production of biodiesel and renewable fuel.

### BACKGROUND OF THE INVENTION

The best known renewable fuels today are ethanol and biodiesel. In the last several years, ethanol has also been blended with gasoline in many metropolitan areas across the country. About half of the gasoline used today in the United States is blended with ethanol at levels of up to 10% by volume (this is called “E10”). Ethanol blends at higher volumes, such as 85% (“E85”), are available in some areas for use in specially designed “flexible-fuel vehicles.” Generally, biodiesel is defined as a fuel having monoalkyl esters of long chain fatty acids derived from plant or animal matter, which meets the requirements of both the United States Environmental Protection Agency and the ASTM.

A third fuel commonly discussed is renewable diesel, which is a broad term that generally encompasses fuels made from biomass feed, including both oils or animal fats, but which are processed through chemical processes which cause hydrogenation of the molecules. Typical among these processes is the replacement of sulfur, oxygen, and nitrogen with hydrogen which converts the triglyceride molecules into paraffinic hydrocarbons. This process is also defined by the IRS as the “thermal depolymerization of oil.”

Other methods of fuel production being researched include biomass to liquid processes and thermal conversion processes. Biomass to liquid processes use high temperature gasification of biomass and a Fischer-Tropsch process to catalytically convert the syngas to liquid fuel. The latter converts biomass or other carbonaceous material into a “bio-oil” which is then refined into a biodiesel fuel.

Moreover, “biodiesel” is generally the name for a variety of ester-based oxygenated fuels made from vegetable oils, fats, greases, or other sources of mono/di/triglycerides. It is a nontoxic and biodegradable substitute and supplement for petroleum diesel. Most biodiesel is produced by the process of acid or base catalyzed transesterification. The transesterification process is a low temperature, low pressure (20 psi) reaction having a high conversion factor (e.g. 98%) with minimal side reactions and reaction time. A fat or oil is reacted with an alcohol (such as methanol or ethanol) in the presence of a catalyst to produce glycerin and alkyl esters, the latter of which comprises biodiesel. The alcohol is charged in an excess stoichiometric amount to drive the reaction and is recovered for reuse. The catalyst is typically sodium or potassium hydroxide which is mixed with the alcohol prior to the transesterification reaction. The biodiesel is then separated from the glycerin with glycerin as a by-product.

Even in blends as low as 20% (B20), biodiesel blends can substantially reduce the emission levels and toxicity of diesel exhaust. Biodiesel has been designated as an alternative fuel by the United States Department of Energy and the United States Department of Transportation, and is registered with the United States Environmental Protection Agency as a fuel and fuel additive. It can be used in any diesel engine, without the need for mechanical alterations, and is compatible with existing petroleum distribution infrastructure.

Conventional biodiesel production systems are based upon large, fixed base plants which require expensive capitalization and on site construction. For example, in order to generate an economically viable amount of biodiesel product, a conventional biodiesel plant contains large, batch-type reactors, large separation units (e.g., decanters, centrifuges, clarifiers), and distillation columns as tall as 50 to 200 feet or more.

### SUMMARY OF THE INVENTION

The present invention includes a process for the production of fuel including the step of introducing an effluent stream into a first vessel, the effluent stream containing solids and at least one of vegetable oil and petroleum oil. The process further includes the steps of heating the effluent to lower its viscosity in the first vessel, separating at least some of the solids from the heated effluent to form a reactant stream, introducing the reactant stream to a bioreactor, introducing at least one remediation agent into the bioreactor, heating the reactor contents to a reaction temperature, and allowing sufficient time to pass such that the reactant stream yields a fuel product. Also included in an aspect of the present invention is the use of the product fuel to fuel an on-site engine.

### BRIEF DESCRIPTION OF THE FIGURES

FIG. 1 illustrates a general process flow diagram in accordance with the present invention and includes the initial steps of the process, including initial effluent treatment;

FIG. 2 illustrates a general process flow diagram in accordance with the present invention and includes intermediate treatment steps prior to bioreactor charging;

FIG. 3 illustrates a general process flow diagram in accordance with the present invention and includes additional treatment steps prior to bioreactor charging; and

FIG. 4 illustrates a general process flow diagram in accordance with the present invention and includes biogrowth steps and bioreactor charging and fuel production.

### DETAILED DESCRIPTION OF THE INVENTION

As noted above, renewable fuels are defined as fuels produced from plant or animal products or wastes, rather than from fossil fuels. Typical today of these types of fuels are biodiesel and renewable diesel. Each is defined generally by the exact nature of the product, which is a result of the method used to create it. Biodiesel can refer to a blend of a petoleum-produced diesel with some amount of renewable diesel or biodiesel. Biodiesel itself, though, is essentially a fatty acid methyl ester and is defined functionally in ASTM D6751 (see ASTM Active Standard: D6751-07be1—Standard Specification for Biodiesel Fuel Blend Stock (B100) for Middle Distillate Fuels). It has most typically been produced by tranesterification of vegetable oil or animal fats.

More specifically, the present invention provides a process that will simultaneously remediate waste or virgin vegetable or petroleum oils while converting those materials into a fuel substitute that can be burned, or combined with a catalyst to produce ethyl or methyl esters of long chain fatty acids (biodiesel), which can be burned. The process remediates vegetable and petroleum oils, removing or breaking down fats, salts, solids and metals using bacteria and enzymes targeted in stages for unwanted pectin, amylases (carbohydrates), lipids, and proteins. The process also allows for the majority of waste removed initially by the system to be converted to alcohol.

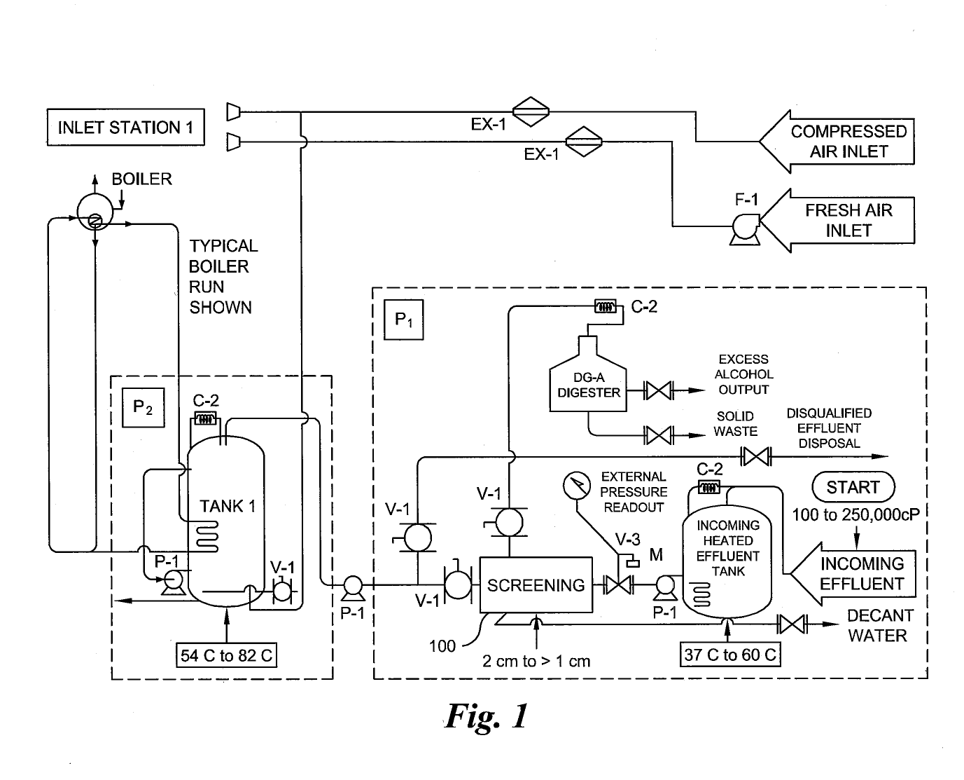

One embodiment of the present invention is partially illustrated in FIG. 1. Incoming material is first heated (to about 37° C. to about 60° C.) to reduce its viscosity and remove water as necessary. The material is then passed through a solids separating means, such as a rotary screen or sieve system 100. Sieve system 100 is sized to separate solids from about 2 cm to about 1 cm. This step removes any large undesired solids, if present. These solids may be sludge, food waste, larger metal items, paper, wood, and other municipal solid wastes. Some of these solids can be sent to a digester reactor and inoculated with bacteria and enzymes in accordance with the present invention to produce alcohol and solid waste byproduct. All expended wastes are dried and disposed of as required by law.

The screened material is then pumped to tank 1, which is preferably a stainless steel, carbon steel, fiberglass, concrete or plastic tank, (depending on need, cost, environmental stresses or jurisdictional regulations). Tank 1 is heated to from about 54° C. to about 82° C. and preferably continuously aerated, such as with a pneumatic sparger system located at the bottom of the tank. The tank contents are preferably blended by a fluid pump of sufficient size to maintain even heat and desired flow characteristics. Tank 1 is sized based on the desired storage and processing ratios, but can be from as small as 500 ml, (for laboratory use), up to 250,000 liters or more.

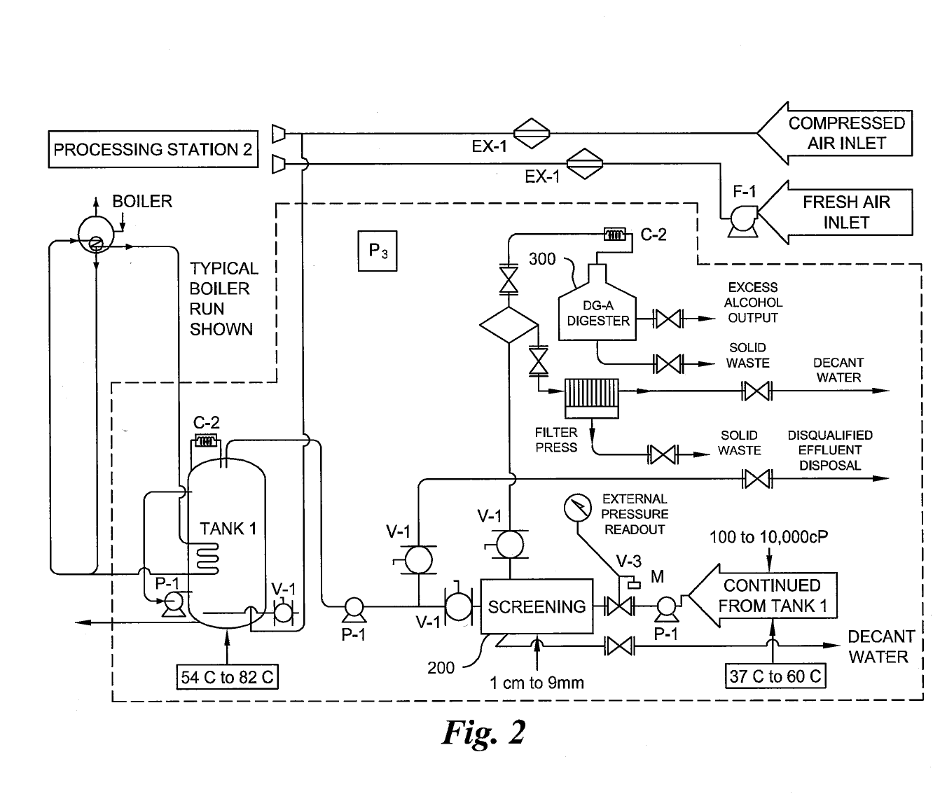

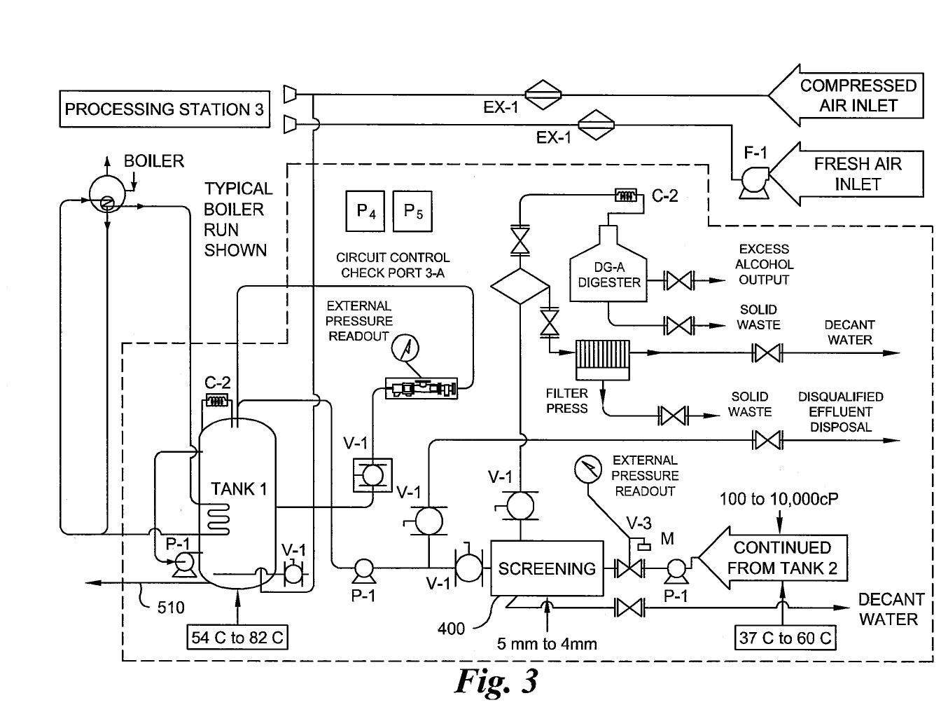

The heated material (preferably at about 37° C. to about 60° C.) showing suitable flow characteristics (i.e., about 100 to about 10,000 cP), is then further classified through a second rotary screen or sieve system 200, (1 cm to 9 mm) that removes any further undesired solids, if present. These solids, which may again be sludge, food waste, metal items, paper wood, and other municipal solid wastes, may optionally be diverted to a digester reactor to form alcohol and solid waste. As shown in FIG. 2, digester 300 receives a stream from the sieve system 200, for biological processing which produces alcohol and solid waste. All expended wastes from digester 300 are dried and disposed of as required. Additional heating and storage steps, and filtering/screening, can occur as necessary to achieve a fluid which is ready to enter a biological reactor tank.

Where additional processing/storage is desired, such as which is illustrated in FIG. 3, screening can occur at even smaller sizes, such as sieve system 400 set at 5 mm to 4 mm. Ultimately, after appropriate treatment and storage as necessary to control fuel production, stream 510 is ready to be processed in bioreactor 4.

The material sent to the biological reactor is allowed to settle. In this embodiment, biological reactor tank 4 can be manufactured from stainless steel, carbon steel, fiberglass, concrete or plastic tank. Tank 4 is heated to a temperature that will cause a desired reaction with a remediation agent (e.g., bacteria) and enzymes to propagate, or be eliminated, based on the desire of the operator, and the desired product. Essentially, at this point, a sample is taken, either manually or automatically, every 5 to 10 minutes and measured for total dissolved solids by electrical conductive or gravimetric means. The fluid must pass through a filter of less than 20 micrometers, and preferably less than 5 micrometers. When this benchmark is reached the fluid is passed through the ultraviolet loop while the fluid is heated to greater than 82° C. to stop biological activity. Once the desired material flow characteristics, moisture, and solids content are determined from samples at sample ports, the material can be adjusted by adding water or other dilution materials.

The source of these bacteria and enzymes can be from any of a number of places. In one embodiment, various concentrations of aerobic and anaerobic bacteria and necessary enzymes are grown and maintained in bioreactors on site. These smaller reactors maintain desired amounts of aerobic, anaerobic and facultative bacteria. Additional reactors can maintain desired amounts of bacterial formulations. One embodiment of the method of the invention for remediation (bioreactor activity) of the material stream comprises the step of dispensing or injecting, from an inoculator apparatus, water-dispersible or water-emulsifiable remediation agents in liquid or dry form that may include vegetative microorganisms, cells, enzymes, spores, bacteria cultures, algae, fungi, nutrients, bacteriophages, buffer salts, activators, surfactants, detergents, lipids, carbohydrates, pectin, proteins, or combinations thereof.

In another embodiment, commercially produced bacteria and enzymes, which are readily available, can be used. Two bacteria, Escherichia coli (E. coli) and or Zymomonas, (including Zymomonas mobilis), and several biologically produced catalysts, in the form of enzymes, can be used. Preferable remediation agents in accordance with the present invention include bacillus, cellulomonas, zymomonas, saccharomyces, aspergillus, trichoderma, escherichia coli, and appropriate combinations thereof.

Cultivation of various species of bacteria can be done through the use of multiple bioreactors, depending on genus, species, and desired loading of the bioreactors during fuel production. A specific example would include the growth and harvesting of Bacillus subtilis, Bacillus amyloliquefaciens, Bacillus lichenformis, and other types of desired bacteria. In such a case, the three species can be grown by themselves, or they can be introduced as a combined product, containing all three species. Commercially available sources would include Biotize, Catalina BioSolutions, and Walling Bio-Act. Some of the commercially available products are produced with engineered nutrients, for growth and cultivation. Bacillus megaterium, Bacillus polymyza, as well as Bacillus cellulomonas, Zymomonas, Zymomonas mobilus, and Escherichia coli are all beneficial bacteria for the process. All strains are non-pathogenic.

An example of a bioreactor solution for the on-sight growth of remediation material (growth media) would consist of peptone, meat extract, and distilled water, that is adjusted to a neutral pH and sterilized. Growth medias (nutrient broth), and other types of bacteria growth media are commercially available.

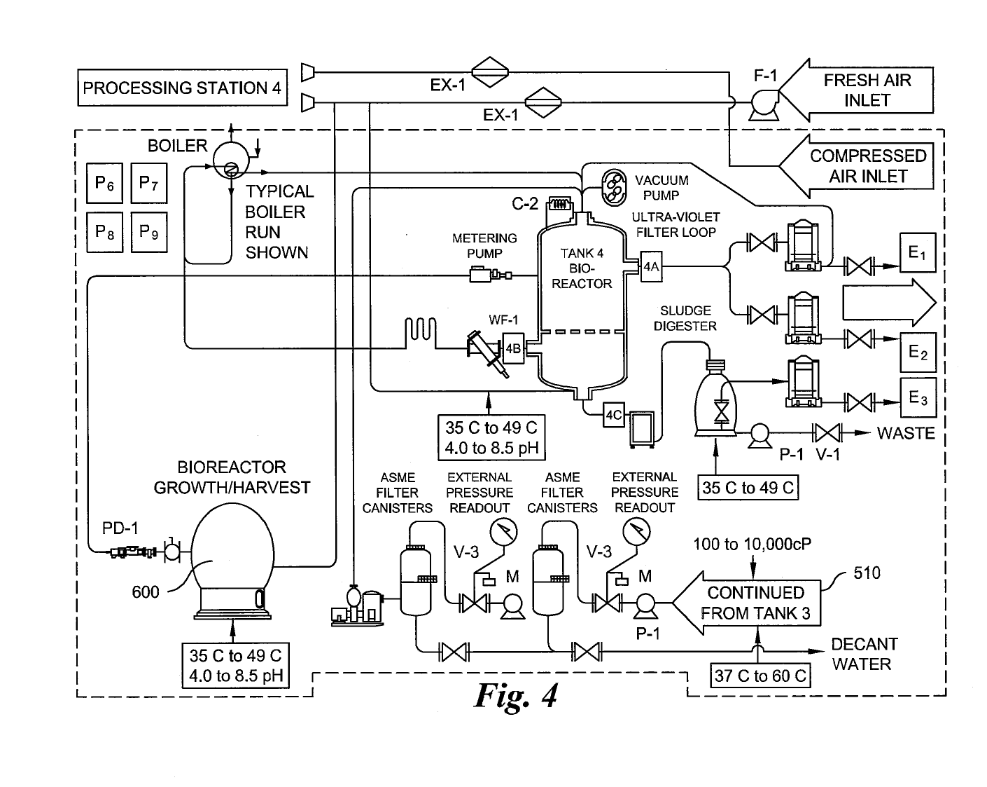

In one embodiment, as shown in FIG. 4, a bio-growth tank 600 is present in conjunction with bioreactor tank 4. In one embodiment, bio-growth tank 600 is filled approximately 50% with growth media. Such a tank is typically 10 to 2,000 liters, but can be larger. The bio-growth tank is controlled to maintain the media temperature within the best range to promote bacteria population growth given the strain involved. Typically this is about 35° C. to about 49° C. An air sparger is preferably constructed in the bottom of the reactor to increase dissolved oxygen. Typically, the bacteria is allowed to propagate until the population is assayed at greater than 25 million cells/gm to 100 million cells/gm. This is determined via common assay methods. The bio-growth tank can preferably have an additional positive displacement pump that is connected to a filter box that contains a 0.45 micron or smaller diaphragm filter that allows for the separation of the bacteria from the majority of the growth media. After adequate reaction, the growth media is diverted to the bioreactor where it will be in continuous contact with the oil containing particulate matter in accordance with the process of the present invention as herein described.

Moreover, and as noted above, the bacteria and enzymes are used to remove unwanted contaminants through metabolic activity, and convert unwanted contaminant substances to useful substances. A specific example of this is glycoside hydrolyse enzyme (Amylase) that hydrolyzes complex carbohydrates into glucose. Glucose and other sugars can be metabolized into alcohol with the use of Escherichia coli (E. coli) and or Zymomonas. Alcohol is desirable and an advantageous substance in the process, in part because it can be used later should a subsequent transesterification process be carried out. Preferably, a pH of 4.0 to 8.5 is maintained in the bioreactor. Operating temperature within biological reactor tank 4 can be rapidly changed, preferably with a heat exchanger. The standard operating temperature within the bioreactor is preferably 35° C. to 49° C. When operating temperature is reached, and inoculation is completed, the mixing pump is turned off, and the biological reactor tank 4 is allowed to settle. Preferably, the anaerobes are present throughout the batch processing time in tank 4.

Biological reactor tank 4 is also preferably equipped with an ultra violet light source. As the operator or control system determines that the material is optimally cleaned, and the bacteria and enzymes have completed the remediation, the contents of the reactor are separated through any suitable and known filtering apparatus. UV light is used to end any biological activity.

The contents of biological reactor tank 4 are then raised to a temperature of at least 82° C. for a period of 5 minutes to kill off any remaining organisms and thereby cease further reaction. In a preferred embodiment, the biological reactor tank contains a simple condenser at the top to allow heat removal, without losing alcohol content. The biological reactor tank contents are then allowed to cool to 37° C., allowing all remaining water to collect at the bottom of the tank for decanting to water recycling tanks. Upon successful decanting, the product fuel is heated again to 82° C. for a period of 5 minutes, and then pumped through a final filter set of 100, 50, and 20 microns. The resulting filtered product fuel is stored in a storage tank, which contains a sparger system which allows for oxygenation prior to use.

At the option of the producer, the product fuel can be diverted for the production of ASTM 6571 biodiesel. The product fuel produced contains an amount of alcohol, from the bioreactor process. The total content of the alcohol can be identified by several common laboratory procedures. The invention conserves capital due to the fact that during the bioreactor process in biological reactor tank 4, the bacteria produces ethanol as a by-product. Depending on the reaction desired, the operator can add additional alcohol, along with sodium hydroxide, sodium methylate, or potassium hydroxide to cause a full reaction to produce ethyl esters and glycerin. The desire to convert the product fuel to biodiesel may be economic, based on the fact that biodiesel may yield a higher selling price in the open marketplace, or be for mechanical performance reasons. The lighter ethyl esters will burn cleaner and produce more energy in a specific engine, over the use of the pure product fuel alone.

At this point, the product fuel can be routed to a conditioning manifold that allows the flow of the product fuel directly to an engine, with or without controlled addition of additives to the product fuel. Additional materials or additives that may be blended in along a conditioning manifold include: glycerin, naphtha, diesel fuel, lubricants, and oxygenate. The conditioning manifold would preferably contain one or more static mixers within the assembly.

Prior to entering the combustion chamber of a reciprocating or turbine engine, where incorrect fuel properties can cause damage, control systems can be in place to insure proper blending and performance. In such an embodiment, the conditioning manifold can contain one or more static mixers within the assembly, with control monitors to measure:

1) Viscosity: This is accomplished by the use of a continuous viscosity monitor. An example is the PSPI Continuous Viscometer is a continuous, on-stream process analyzer for measurement of the absolute viscosity of a fluid. This is a unique application of viscosity measurement in that it occurs just prior to the fuel or blend entering the combustion chamber, or into a pre-combustion storage (buffer) tank. In such a case, the control system would be programmed to shut down fuel feed if the measured viscosity is outside of a predetermined range.

2) Sulfur: An example of this is the use of near instant (one minute or less) sample tester for sulfur that would be used within the conditioning manifold. A specific example would be the PAC 6000 SERIES PROCESS/ON-LINE SULFUR/NITROGEN ANALYZER. This equipment, or similar equipment, can identify within a minute or less the sulfur content of the fuel in route to the combustion chamber of the engine.

3) Particulates: Near instantaneous on-line testing equipment exists for fluids. The unique conditioning manifold allows the continuous monitoring of the fuel mixture as it travels to the combustion chamber, either directly or held in a pre-combustion storage (buffer) tank. This unique application allows for the suspension of the operating engine if the particulate level is out of acceptable range which could cause significant engine damage.

Furthermore, the present invention includes the aspect that the operator can produce the pure product fuel alone, or an ASTM Standard biodiesel in the same system, in whole or in part, solely at the discretion of the operator. After production of the product fuel, additional possible materials may be produced, including biodiesel and glycerin (waste from biodiesel reaction) which can then be stored separately in tanks.

More specifically, two fuel blends or products may be created from the product fuel. The first is the result of a short reaction of the product fuel to form small amounts of mono-alkyl esters of long chain fatty acids within the fuel, and glycerin, through the starving of catalyst during the process an one or more additives. The second would include the fully catalyzed product fuel to provide complete reaction to mono-alkyl esters of long chain fatty acids.

A preferred embodiment includes a control system that contains three programmable controllers that monitor the system at each critical stage for temperature, viscosity, processing time, water content, particulate content, inoculation time and volume, tank transfer, pump operation, heating, cooling, filling, emptying, emergency tank evacuation, fire control, additive control, blending, storage, combustion feed, engine start, engine shutdown, vacuum, pressure, sulfur content, reaction checks, oxygen content, oxygenation, filtering, waste removal, video, and audio surveillance, theft, and remote reporting.

As noted above, there are several places in the process where a digester can be used. These units are standard and available commercially. Generally, this unit operation employs microorganisms to convert waste water to a readily disposable digested sludge. Anaerobic digestion is a bacterial process that breaks down organic materials within waste in the absence of oxygen. It is generally run in closed tanks. Generally, biomass processing waste is mixed with water and fed into the digester without air.

During anaerobic digestion, materials are segregated in a variety of ways. Some relatively light materials entrap rising gas bubbles and are transported to the liquid surface in the digester. Similarly, some of the microscopic biomass in raw sludge retains microscopic bubbles and is transported to the surface. Other materials having a specific gravity less than the digester liquid in which they are suspended rise through natural buoyancy.

In another embodiment of the invention, the fuel product can be further processed. One such further processing step would include transesterification. As noted above, this step can be used at any appropriate point in the process. In other words, once the processed fluid meets the total dissolved solids requirements, some or all can be diverted or transferred to a tank where alcohol can be added with a caustic and reacted to produce biodiesel. One on site production benefit is seen in this context as the glycerin can be used as a fuel dilutant, as opposed to paying for its disposal as a byproduct.

One of the advantages of the present invention is that it can provide a source of on-site fuel generation for remote turbine or engine operation. This advantage is particularly realized where remote areas have grease or waste oil sources that would otherwise need to be shipped great distances for treatment. By providing an operation that produces fuel on-site, power can be generated with minimal transportation costs associated with the power generation.

### EXAMPLE 1

A 2,500 gallon sample load of waste vegetable oil was initially sampled and found to contain particulate, sludge and water contamination of 22%, 4%, and 11% respectively, by volume. The sample load was air blended (sparger) within a 3,500 gallon tank for 30 minutes prior to the taking of samples. The sample load was processed through an initial 0.250 sieve screen, approximately 25% of the total 26% solids by volume were removed by the initial screen. The material was then heated to 140° F. The material was then allowed to settle in a 2,500 gallon high density polyethylene tank for 3 hours for decanting preparation. Excess water was then drained from the bottom of the tank. Nearly all of the 11% by volume of water was extracted at that point.

The remaining material was pumped into a 5,000 gallon heating vessel and heated to 40° C. by alloy electrical immersion heaters built into a self contained heat exchanger consisting of a 3-inch galvanized pipe.

Two pounds of facultative anaerobes and aerobic bacteria, nutrient and enzymes, supplied by Catalina Bio Solutions in the commercial form of “BioTreatment System” was introduced into the tank at temperature. The bacteria and enzyme solution, which is typically engineered to eliminate all particulates including fats and oils, was monitored every 4 hours by taking a hand sample of 250 ml from the tank at the bottom. The tank was under constant flow mixing to the top from the bottom through the heat exchanger.

At 30 minutes, a 250 ml sample was taken, and the amount of visible particulates was approximately 2% sludge, 10% particulates, and an increase of 3% water.

At 4 hours and 30 minutes a 250 ml sample was taken, and the amount of visible particulates was approximately 2% sludge, 6% particulates, and a decrease to less than 1% water.

At 9 hours a 250 ml sample was taken, and the amount of visible particulates was approximately 3% sludge, 3% particulates, and less than 1% water.

At 9 hours the material was processed through a series of 0.50 filters and then high temperature processed at 85° C. to drive off any excess water and residual alcohols.

The remaining material, now approximately 1,550 gallons a fuel, was burned in a 25 Kilowatt reciprocating diesel laboratory generator for approximately 705 hours.

### EXAMPLE 2

A 1,000 gallon sample load of waste vegetable oil, mixed with interceptor waste water was initially sampled as described and found to contain particulate, sludge and water contamination of 31%, 9%, and 18% respectively, by volume. The sample load was air blended (sparger) within a 3,500 gallon tank for 30 minutes prior to the taking of samples. The sample load was processed through an initial 0.250 sieve screen. Approximately 50% of the total solids by volume were removed by the initial screen. The material was then heated to 140° F., and sent through a series of vertical screen canisters, containing a 0.125 screen, a 20 mesh screen, and an 80 mesh screen. The remaining material was allowed to settle in a 2,500 gallon high density polyethylene tank for 24 hours in preparation for decanting. Excess water was then drained from the bottom of the tank. All of the visible water was extracted.

The remaining material was pumped into a 5,000 gallon heating vessel and heated to 40° C. by alloy electrical immersion heaters built into a self contained heat exchanger consisting of 3-inch galvanized pipe.

One pound of a powder combination containing: 25% non-pathogenic Bacillus subtillus and Escherichia coli, and the following enzymes: protease, pectinase, amylase, cellulose, and lipase, all at 15% by weight of the total, was prepared. A separate nutrient system was dissolved in five gallons of distilled water, and then the one pound of powder containing the bacteria and enzymes was added. This mixture was allowed to stand under ambient room temperature at about 24° C. for 24 hours and then added into the tank at temperature. The bacteria and enzyme solution, was monitored every 2 hours by taking a hand sample of 500 ml from the tank at the bottom, mid-point, and top. The tank was under constant flow mixing to the top from the bottom through the heat exchanger.

At 4 hours a 500 ml sample was taken from the bottom of the tank, and the amount of visible particulates was approximately 1% sludge, 8% particulates, and an increase to 4% water.

At 4 hours a 500 ml sample was taken from the top of the tank, and the amount of visible particulates was approximately 1% sludge, 8% particulates, and an increase to 4% water (basically the same as the sample from the bottom of the tank). The mid-point sample failed to flow properly.

At 8 hours a 500 ml sample was taken, and the amount of visible particulates was approximately >1% sludge, 2% particulates, and no visible water.

A sample from the top of the tank produced similar results.

A 50 cc syringe sample from the mid point of the tank produced similar results.

At 24 hours a 500 ml sample was taken, from the top and bottom of the tank. The amount of visible particulates was approximately >1% sludge, particulates were visible in a light white film throughout the material, and no visible water was observed.

At 24 hours the material was processed through a diaphragm mounted on a 0.45 micron filter and then high temperature processed at 85° C. to drive off any excess water and residual alcohols. About 3% sludge material was filtered out, made up of a white foamy material and a dark brown material.

The remaining material, now a fuel, was burned in a 25 Kilowatt reciprocating diesel laboratory generator.

Representative, commercially available bacteria packages include:

Commercial Brands

| Sample | | | |
|---|---|---|---|
| Number | Brand: | Manufacturer: | Type: |
| 1 | Sporezyme | Walling | Proprietary |
| 2 | Biotize | Hagen | Proprietary |
| 3 | Pectinex | | Proprietary |
| 4 | Efinol L | Prokura | Proprietary |
| 5 | Ultrazyme | Cypher | Proprietary |
| 6 | Termamyl | | alpha amylase |
| 11 | AMG | | amyloglucosidase |
| 15 | Neutrase | | amyloliquefaciens |
| 18 | Aluminate | Thatcher Chem | Flocculent |
| 19 | Celluclast | | Cellulase |

What is claimed:

1. A process for the production of fuel comprising the steps of:
introducing an effluent stream into a first vessel, the effluent stream containing solids and at least one of vegetable oil and petroleum oil;
heating the effluent to lower its viscosity in the first vessel;
separating at least some of the solids from the heated effluent to form a reactant stream;
introducing the reactant stream to a bioreactor;
introducing at least one remediation agent into the bioreactor;
heating the reactor contents to a reaction temperature;
allowing sufficient time to pass such that the reactant stream yields a fuel product.

2. The process of claim 1 wherein the remediation agent is selected from the group consisting of bacillus, cellulomonas, zymomonas, saccharomyces, aspergillus, trichoderma, escherichia coli, and combinations thereof.

3. The process of claim 1 wherein the fuel product is further mixed with an additive selected from the group consisting of: viscosity modifiers, naptha, lubricants, diesel, Jet A, and oxygenates.

4. The process of claim 1 further comprising the step of transesterification of the fuel product.

5. The process of claim 1 further comprising the step of obtaining the effluent stream from a municipal grease trap.

6. A process for powering an engine comprising the steps of:
introducing an effluent stream into a first vessel, the effluent stream containing solids and at least one of vegetable oil and petroleum oil;
heating the effluent to lower its viscosity in the first vessel;
separating at least some of the solids from the heated effluent to form a reactant stream;
introducing the reactant stream to a bioreactor;
introducing at least one remediation agent into the bioreactor;
heating the reactor contents to a reaction temperature;
allowing sufficient time to pass such that the reactant stream yields a fuel product;
passing the fuel product to an on-site engine.

7. The process of claim 6 wherein the remediation agent is selected from the group consisting of bacillus, cellulomonas, zymomonas, saccharomyces, aspergillus, trichoderma, escherichia coli, and combinations thereof.

8. The process of claim 6 wherein the fuel product is further mixed with an additive selected from the group consisting of: viscosity modifiers, naptha, lubricants, diesel, Jet A, and oxygenates.

9. The process of claim 6 further comprising the step of transesterification of the fuel product before it is passed to the on-site engine.

10. The process of claim 6 further comprising the step of obtaining the effluent stream from a municipal grease trap.
